(19) United States
(12) Patent Application Publication
(10) Pub. No.: US 2004/0180918 A1
(43) Pub. Date: Sep. 16, 2004

(54) Novel therapeutic method

(76) Inventors: Richard Graham Knowles, Stevenage Hertfordshire (GB); Peter Ward, Stevenage Hertfordshire (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/484,292

(22) PCT Filed: Jul. 25, 2002
(86) PCT No.: PCT/EP02/08322

(30) Foreign Application Priority Data
Jul. 27, 2001 (GB) 0118373.0

(51) Int. Cl. A61K 31/4745; A61K 31/47

(52) U.S. Cl. 514/291; 514/311

(57) ABSTRACT

This invention relates to treating pulmonary diseases such as obstructive pulmonary disease or asthma by administering a phosphodiesterase 4 inhibitor in combination with an anticholinergic agent.

### AREA OF THE INVENTION

This invention relates compositions and methods for preventing or reducing the onset of symptoms of pulmonary diseases, or treating or reducing the severity of pulmonary diseases. In particular it relates to compositions and methods for treating pulmonary diseases by administering a PDE 4 inhibitor and an anticholinergic agent, particularly an M₁, M₂, M₁/M₂ or M₃ receptor antagonist.

### BACKGROUND OF THE INVENTION

Identification of novel therapeutic agents for treating pulmonary diseases is made difficult by the fact that multiple mediators are responsible for the development of the disease. Thus, it seems unlikely that eliminating the effects of a single mediator could have a substantial effect on all other components of a particular pulmonary disease. An alternative to the “mediator approach” is to regulate the activity of the cells responsible for the pathophysiology of the disease. That approach as set forth in this invention utilizes two regulators, a PDE4-specific inhibitor and an anticholinergic agent.

PDE4-specific inhibitors represent a new approach to cell regulation by elevating levels of cAMP (adenosine cyclic 3′,5′-monophosphate). Cyclic AMP has been shown to be a second messenger mediating the biologic responses to a wide range of hormones, neurotransmitters and drugs; [Krebs Endocrinology Proceedings of the 4th International Congress Excerpta Medica, 17-29, 1973]. When the appropriate agonist binds to specific cell surface receptors, adenylate cyclase is activated, which converts Mg⁺²-ATP to cAMP at an accelerated rate.

Cyclic AMP modulates the activity of most, if not all, of the cells that contribute to the pathophysiology of extrinsic (allergic) asthma. As such, an elevation of cAMP should produce beneficial effects including: 1) airway smooth muscle relaxation, 2) inhibition of mast cell mediator release, 3) suppression of neutrophil degranulation, 4) inhibition of basophil degranulation, and 5) inhibition of monocyte and macrophage activation. Hence, compounds that activate adenylate cyclase or inhibit phosphodiesterase should be effective in suppressing the inappropriate activation of airway smooth muscle and a wide variety of inflammatory cells. The principal cellular mechanism for the inactivation of cAMP is hydrolysis of the 3′-phosphodiester bond by one or more of a family of isozymes referred to as cyclic nucleotide phosphodiesterases (PDEs).

It has been shown that a distinct cyclic nucleotide phosphodiesterase (PDE) isozyme, PDE 4, is responsible for cAMP breakdown in airway smooth muscle and inflammatory cells. [Torphy, “Phosphodiesterase Isozymes: Potential Targets for Novel Anti-asthmatic Agents” in New Drugs for Asthma, Bames, ed. IBC Technical Services Ltd., 1989]. Research indicates that inhibition of this enzyme not only produces airway smooth muscle relaxation, but also suppresses degranulation of mast cells, basophils and neutrophils along with inhibiting the activation of monocytes and neutrophils. Moreover, the beneficial effects of PDE 4 inhibitors are markedly potentiated when adenylate cyclase activity of target cells is elevated by appropriate hormones or autocoids, as would be the case in vivo. Thus PDE 4 inhibitors, and particularly PDE4-specific inhibitors, would be effective in the lung, where levels of prostaglandin E₂ and prostacyclin (activators of adenylate cyclase) are elevated.

In addition, it could be useful to combine therapies, in light of the fact that the etiology of many pulmonary diseases involves multiple mediators. In this invention there is presented the combination of a PDE 4 inhibitor and an appropriate anticholinergic agent for treating pulmonary diseases, particularly chronic obstructive pulmonary disease (COPD), asthma or a related pulmonary disease such as chronic bronchitis or allergic rhinitis.

### SUMMARY OF THE INVENTION

In a first aspect this invention relates to a method of prophylaxis of, treating, or reducing the exacerbations associated with, a pulmonary disease by administering to a patient in need thereof an effective amount of a PDE 4 inhibitor and an anticholinergic agent either in a single combined form, separately, or separately and sequentially where the sequential administration is close in time, or remote in time.

In a second aspect this invention relates to a composition for the prophylaxis of, treating, or reducing the exacerbations associated with, a pulmonary disease comprising an effective amount of a PDE4 inhibitor, an effective amount of an anticholinergic agent, and a pharmaceutically acceptable excipient.

In a third aspect this invention relates to a method for preparing a composition which is effective for the prophylaxis of, treating, or reducing the exacerbations associated with, a pulmonary disease which method comprises mixing an effective amount of a PDE4 inhibitor and an anticholinergic agent with a pharmaceutically acceptable excipient.

In a fourth aspect there is provided use of an effective amount of a PDE 4 inhibitor and an anticholinergic agent either in a single combined form, separately, or separately and sequentially where the sequential administration is close in time, or remote in time in the manufacture of a medicament or medicament pack for the prophylaxis of, treating, or reducing the exacerbations associated with, a pulmonary disease.

In a fifth aspect there is provided use of a composition comprising an effective amount of a PDE4 inhibitor, an effective amount of an M₁, M₂ or M₁/M₂ receptor antagonist and a pharmaceutically acceptable excipient in the manufacture of a medicament for the prophylaxis of, treating, or reducing the exacerbations associated with, a pulmonary disease.

### DETAILED DESCRIPTION OF THE INVENTION

The combination therapy contemplated by this invention comprises administering a PDE4 inhibitor with an anticholinergic agent, particularly an M₁, M₂ or M₁/M₂ receptor antagonist, to prevent onset of a pulmonary disease event, to treat an existing condition, or to reduce the frequency or severity of exacerbations often occurring in patients suffering from a chronic respiratory disease. The compounds may be administered together in a single dosage form. Or they may be administered in different dosage forms. They may be administered at the same time. Or they may be administered either close in time or remotely, such as where one drug is administered in the morning or the second drug is administered in the evening. The combination may be used prophylactically or after the onset of symptoms has occurred. In some instances the combination(s) may be used to prevent the progression of a pulmonary disease or to arrest the decline of a function such as lung function. In addition, this combination is useful for reducing the incidences and/or severity of exacerbations of some pulmonary diseases, such as COPD. See co-pending U.S. provisional application 60/221,275 filed 27 Jul. 2000 for test methods for determining and evaluating the affects of this combination on the frequency and severity of exacerbations in COPD patients. That methodology, and the full disclosure of that application, is incorporated herein in full as if set forth herein.

The PDE4 inhibitor useful in this invention may be any compound that is known to inhibit the PDE4 enzyme or which is discovered to act in as PDE4 inhibitor, and which is only or essentially only a PDE4 inhibitor, not compounds which inhibit to a degree of exhibiting a therapeutic effect other members of the PDE family as well as PDE4. Generally it is preferred to use a PDE4 antagonists which has an IC₅₀ ratio of about 0.1 or greater as regards the IC₅₀ for the PDE 4 catalytic form which binds rolipram with a high affinity divided by the IC₅₀ for the form which binds rolipram with a low affinity.

PDE inhibitors used in treating inflammation and as bronchodilators, drugs like theophylline and pentoxyfyllin, inhibit PDE isozymes indiscriminently in all tissues. These compounds exhibit side effects, apparently because they non-selectively inhibit all 5 PDE isozyme classes in all tissues. The targeted disease state may be effectively treated by such compounds, but unwanted secondary effects may be exhibited which, if they could be avoided or minimized, would increase the overall therapeutic effect of this approach to treating certain disease states. For example, clinical studies with the selective PDE 4 inhibitor rolipram, which was being developed as an antidepressant, indicate it has psychotropic activity and produces gastrointestinal effects, e.g., pyrosis, nausea and emesis.

It turns out that there are at least two binding forms on human monocyte recombinant PDE 4 (hPDE 4) at which inhibitors bind. One explanation for these observations is that hPDE 4 exists in two distinct forms. One binds the likes of rolipram and denbufylline with a high affinity while the other binds these compounds with a low affinity. The preferred PDE4 inhibitors of for use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE 4 catalytic form which binds rolipram with a high affinity divided by the IC₅₀ for the form which binds rolipram with a low affinity.

Reference is made to U.S. Pat. No. 5,998,428, which describes these methods in more detail. It is incorporated herein in full as though set forth herein.

Most preferred are those PDE4 inhibitors which have an IC₅₀ ratio of greater than 0.5, and particularly those compounds having a ratio of greater than 1.0.

Preferred compounds are cis [cyano-4-3-cyclopentyloxy4-methoxyphenyl)cyclohexan-1-carboxylate] also known as cilomilast or Ariflo®, 2-carbomethoxy4-cyano-4-4-(3-cyclopropylmethoxy-4-difluoromethoxyphenyl)cyclohexan-1-one, and cis [4-cyano-4-(3-cyclopropylmethoxy-4-difluoromethoxyphenyl)cyclohexan-1-ol]. They can be made by the processed described in U.S. Pat. Nos. 5,449,686 and 5,552,438. Other PDE4 inhibitors, specific inhibitors, which can be used in this invention are AWD-12-281 from Astra (Hofgen, N. et al. 15th EFMC Int Symp Med Chem (September 6-10, Edinburgh) 1998, Abst P.98); a 9-benzyladenine derivative nominated NCS-613 (INSERM); D-4418 from Chiroscience and Schering-Plough; a benzodiazepine PDE4 inhibitor identified as CI-1018 (PD-168787; Parke-Davis[Warner-Lambert); a benzodioxole derivative Kyowa Hakko disclosed in WO 9916766; V-1 1294A from Napp (Landells, L. J. et al. Eur. Resp J [Annu Cong Eur Resp Soc (September 19-23, Geneva) 1998] 1998, 12(Suppl. 28): Abst P2393); roflumilast (CAS reference No 162401-32-3) and a pthalazinone (WO 9947505) from Byk-Gulden; or a compound identified as T-440 (Tanabe Seiyaku; Fuji, K. et al. J Pharmacol Exp Ther,1998, 284(1): 162).

The anticholinergic agents of this invention are those compounds that act as antagonists at the muscarinic receptor. These receptors are found primarily on the autonomic effector cells that are innervated by postganglionic parasympathetic nerves. They are also present in the brain, in ganglia, and on some blood cells such as blood vessels. Early work on this type of receptor identified subtypes characterized as being in the periphery and the CNS of cells and tissues. They were differentiated on the basis to two agonist, McN-A-343 and bethanechol and labeled “M₁” (ganglionic) and “M₂” (effector cells). In 1988 Goyal published a review of the then current knowledge of these two receptors (Goyal, R. K., Identification, Localizaton and classification of muscarinic receptor subtypes in the gut. Life Sci. 1988, 43, 2009-2220). Subsequent work using cDNA cloning techniques has identified five distinct subtypes to date (Bonner et al., Science, 1987, 237: 527-531). For the purposes of this treatment methodology, the primary interest is in the M₁ and M₂ receptors and antagonists of these receptors. Exemplary compounds are the alkaloids of the belladonna plants as illustrated by the likes of atropine, scopolamine, homatropine, hyoscyamine; these compounds are normally administered as a salt, being tertiary amines. These drugs, particularly the salt forms, are readily available from a number of commercial sources or can be made or prepared from literature data via, to wit:

Atropine—CAS-51-55-8 or CAS-5148-1 (anhydrous form), atropine sulfate—CAS-5908-99-6; atropine oxide—CAS4438-22-6 or its HCl salt—CAS4574-60-1 and methylatropine nitrate—CAS-52-88-0.

Homatropine—CAS-8700-3, hydrobromide salt—CAS-51-56-9, methylbromide salt—CAS-8049-9.

Hyoscyamine (d, l)—CAS-101-31-5, hydrobromide salt—CAS-306-03-6 and sulfate salt—CAS-6835-16-1.

Scopolamine—CAS-51-34-3, hydrobromide salt—CAS-6533-68-2, methylbromide salt—CAS-155-41-9.

Quaternary ammonium derivatives of the belladonna alkaloids are also useful in this combination. By way of example ipratropium bromide, sold under the name Atrovent is a quaternary ammonium derivative of atropine formed by the introduction of an isopropyl group on the nitrogen of atropine. Another derivative of atropine, oxitropium bromide, has an ethyl group on the nitrogen of the azabicyclo[3.2.1]octyl ring. A related compound is tiotropium (CAS-13940448-1) and its bromide salt (Spiriva®). Also of interest are: methantheline (CAS-5346-3), propantheline bromide (CAS-50-34-9), anisotropine methyl bromide or Valpin 50 (CAS-80-50-2), clidinium bromide (Quarzan, CAS-3485-62-9), copyrrolate (Robinul), isopropamide iodide (CAS-71-81-8), mepenzolate bromide (U.S. Pat. No. 2,918,408), tridihexethyl chloride (Pathilone, CAS4310-354), and hexocyclium methylsulfate (Tral, CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, or methoctramine

These compounds are available through commercial sources. In addition, they are described in some detail in the text Goodman & Gilman's The Pharmacological Basis of Therapeutics, Ninth Ed, 1996, McGraw-Hill at pages 586 to 591 and most are set out in some form or another in The Physicians Desk Reference, (Vol. 54, 2000, Medical Economic Co., Montvale, N.J., USA. Both references provide information about each compound, dosing and routes of administration, with exemplary formulation data, as to the Chemical Abstracts System numbers and the US patent noted for mepenzolate bromide.

One or more of these anticholinergic agents can be use with one or more PDE4 inhibitors for prophylaxis or treatment.

All compounds mentioned may, if desired and appropriate, be employed in the form of alternative pharmaceutically acceptable derivatives, eg. salts and esters thereof.

These drugs are usually administered as an oral preparation or a nasal spray or aerosol, or as an inhaled powder. This invention contemplates either co-administering both drugs in one delivery form such as an inhaler, that is, putting both drugs in the same inhaler. Alternatively one can put the PDE4 inhibitor into pills and package them with an inhaler that contains the anticholinergic.

The present compounds and pharmaceutically acceptable salts, which are active when given orally, can be formulated as syrups, tablets, capsules, controlled-release preparation or lozenges or as an inhalable preparation.

A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical compositions for inhalation are in the form of a dry powder, solution, suspension or emulsion. Administration may for example be by dry powder inhaler (such as unit dose or multi-dose inhaler, e.g. as described in U.S. Pat. No. 5,590,645) or by nebulisation or in the form of a pressurized aerosol. Dry powder compositions typically employ a carrier such as lactose, trehalose or starch. Compositions for nebulisation typically employ water as vehicle. Pressurized aerosols typically employ a propellant such as dichlorodifluoromethane, trichlorofluoromethane or, more preferably, 1,1,1,2-tetrafluoroethane, 1,1,1,2,3,3,3-heptafluoro-n-propane or mixtures thereof. Pressurized aerosol formulations may be in the form of a solution (perhaps employing a solubilising agent such as ethanol) or suspensions that may be excipient free or employ excipients including surfactants and/or co-solvents (e.g. ethanol). In dry powder compositions and suspension aerosol compositions the active ingredient will preferably be of a size suitable for inhalation (typically having mass median diameter (MMD) less than 20 microns e.g. 1-10 especially 1-5 microns). Size reduction of the active ingredient may be necessary e.g. by micronisation.

Pressurized aerosol compositions will generally be filled into canisters fitted with a valve, especially a metering valve. Canisters may optionally be coated with a plastic material e.g. a fluorocarbon polymer as described in WO96/32150. Canisters will be fitted into an actuator adapted for buccal delivery.

Typical compositions for nasal delivery include those mentioned above for inhalation and further include non-pressurized compositions in the form of a solution or suspension in an inert vehicle such as water optionally in combination with conventional excipients such as buffers, anti-microbials, tonicity modifying agents and viscosity modifying agents which may be administered by nasal pump.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

Each dosage unit for oral administration contains suitably from 0.3 mg to 60 mg/Kg, and preferably from 1 mg to 30 mg/Kg of a compound or a pharmaceutically acceptable salt thereof. Preferred doses include 10 mg and 15 mg/Kg for treating COPD. Each dosage unit for parenteral administration contains suitably from 0.1 mg to 100 mg/Kg, of the compound or a pharmaceutically acceptable salt thereof. Each dosage unit for intranasal administration contains suitably 1-400 mcg and preferably 10 to 200 mcg per activation. A topical formulation contains suitably 0.001 to 5.0% of a present compound. The active ingredient may be administered from 1 to 6 times a day, sufficient to exhibit the desired activity. Preferably, the active ingredient is administered once or twice a day.

It is contemplated that both active agents would be administered at the same time, or very close in time. Alternatively, one drug could be taken in the morning and one later in the day. Or in another scenario, one drug could be taken twice daily and the other once daily, either at the same time as one of the twice-a-day dosing occurred, or separately. Preferably both drugs would be taken together at the same time and be administered as an admixture.

The following examples are provided to illustrate how to make and use the invention. They arc not in any way intended to limit the scope of the invention in any manner or to any degree. Please refer to the claims for what is reserved to the inventors hereunder.

### EXAMPLES

The following eight assays spread among five species were used to develop data supporting the selection of an IC₅₀ ratio of about 0.1 or greater. The assays were: stimulation of acid production from rabbit isolated parietal gland; inhibition of FMLP-induced degranulation (release of myleoperoxidase) in human neutrophils; inhibition of FMLP-included O₂⁻ formation in guinea pig eosinophils; inhibition of LPS-induced TNF_{α} production in human monocytes; production of emesis in dogs; inhibition of antigen-induced bronchoconstriction in guinea pigs; reversal of reserpine-induced hypothermia in mice; and inhibition of LPS-induced TNF_{α} production from adoptively-transferred human monocytes in mice. These assays and data are presented below.

Statistical Analysis

To examine the hypothesis that inhibition of the low affinity site PDE 4 is associated with the anti-inflammatory actions of this class of compounds, whereas inhibition of the high affinity site is associated with the production of certain side effects, we determined the ability of various PDE 4 inhibitors to block inflammatory cell function both in vitro and in vivo and the ability of these compounds to produce side effects in in vitro and in vivo models. To compare the ability of PDE 4 inhibitors to elicit a given therapeutic effect or side effect with their ability to inhibit the low affinity binding site versus their ability to inhibit the high affinity site of PDE 4, we compared the potency of these compounds in the in vitro or in vivo assays with their potency against the isolated enzyme catalytic activity or the high affinity site by a linear correlation of (r²) or a rank order correlation (Spearman's Rho). The linear correlation asks whether the potency of a compound at inhibiting either the low affinity site or the high affinity site can be used to predict the ability to elicit a given anti-inflammatory or side effect. The rank order correlation tests whether the rank order potency in producing a given anti-inflammatory or side effect is similar to the rank order potency in inhibiting the low affinity or the high affinity site, Both r² and Spearman's Rho were calculated using the STAT View II computer program for the Macintosh.

### PDE 4 Versus Rolipram High Affinity Binding

### Example 1

### Phosphodiesterase and Rolipram Binding Assays

### Example 1A

Isolated human monocyte PDE 4 and hrPDE (human recombinant PDE4) was determined to exist primarily in the low affinity form. Hence, the activity of test compounds against the low affinity form of PDE 4 can be assessed using standard assays for PDE 4 catalytic activity employing 1 μM [³H]cAMP as a substrate (Torphy et al., J. of Biol. Chem., Vol. 267, No. 3 pp1798-1804, 1992).

Rat brain high-speed supernatants were used as a source of protein. Enantionmers of [³H]-rolipram were prepared to a specific activity of 25.6 Ci/mmol. Standard assay conditions were modified from the published procedure to be identical to the PDE assay conditions, except for the last of the cAMP: 50 mM Tris HCl (pH 7.5), 5 mM MgCl₂, and 1 nanoM of [³H]-rolipram (Torphy et al., J. of Biol. Chem., Vol. 267, No.3 pp1798-1804, 1992). The assay was run for 1 hour at 30° C. The reaction was terminated and bound ligand was separated from free ligand using a Brandel cell harvester. Competition for the high affinity binding site was assessed under conditions that were identical to those used for measuring low affinity PDE activity, expect that [³H]-cAMP and [3H]5′-AMP were not present. The data presented in Table I, page 8 were generated using the protocol described in Example 1A.

### Example 1B

### Measurement of Phosphodiesterase Activity

PDE activity was assayed using a [³H]cAMP scintillation proximity assay (SPA) or [³H]cGMP SPA enzyme assay as described by the supplier (Amersham Life Sciences). The reactions were conducted in 96-well plates at room temperature, in 0.1 ml of reaction buffer containing (final concentrations): 50 mM Tris-HCl, pH 7.5, 8.3 mM MgCl2, 1.7 mM EGTA, [³H]cAMP or [³H] cGMP (approximately 2000 dpm/pmol), enzyme and various concentrations of the inhibitors. The assay was allowed to proceed for 1 hr and was terminated by adding 50 μl of SPA yttrium silicate beads in the presence of zinc sulfate. The plates were shaken and allowed to stand at room temperature for 20 min. Radiolabeled product formation was assessed by scintillation spectrometry. Activities of PDE3 and PDE7 were assessed using 0.05 μM [³H]CAMP, whereas PDE4 was assessed using 1 μM [³H]cAMP as a substrate. Activity of PDE1B, PDE1C, PDE2 and PDE5 activities were assessed using 1 μM [³H]cGMP as a substrate.

[³H]R-Rolipram Binding Assay

The [³H]R-rolipram binding assay was performed by modification of the method of Schneider and co-workers, see Nicholson, et al., Trends Pharmacol Sci., Vol. 12, pp.19-27 (1991) and McHale et al., Mol. Pharmacol., Vol.39, 109-113 (1991). R-rolipram binds to the catalytic site of PDE4 see Torphy et al., Mol. Pharmacol., Vol.39, pp. 376-384 (1991). Consequently, competition for [³H]R-rolipram binding provides an independent confirmation of the PDE4 inhibitor potencies of unlabeled competitors. The assay was performed at 30° C. for 1 hr in 0.5 μl buffer containing (final concentrations): 50 mM Tris-HCl, pH 7.5, 5 mM MgCl₂, 0.05% bovine serum albumin, 2 nM [³H]R-rolipram (5.7×104 dpm/pmol) and various concentrations of non-radiolabeled inhibitors. The reaction was stopped by the addition of 2.5 ml of ice-cold reaction buffer (without [³H]-R-rolipram) and rapid vacuum filtration (Brandel Cell Harvester) through Whatman GF/B filters that had been soaked in 0.3% polyethylenimine. The filters were washed with an additional 7.5-ml of cold buffer, dried, and counted via liquid scintillation spectrometry.

### Formulation Examples

A: Metered Dose Inhalers

TABLE 1

| | Per actuation |
|---|---|
| Cilomilast | 18 mcg |
| Tiotropium bromide | 18 mcg |
| 1,1,1,2-Tetrafluoroethane | to 75.0 mg |

The micronised active ingredients (eg. for 120 actuations) are weighed into an aluminum can, 1,1,1,2-tetrafluoroethane is then added from a vacuum flask and a metering valve is crimped into place.

B: Dry Powder Inhalers

TABLE 2

| | Per cartridge or blister |
|---|---|
| Cilomilast | 150 mcg |
| Tiotropium bromide | 0.4 mcg |
| Lactose Ph. Eur. | to 12.5 mg |

The active ingredients are micronised and bulk blended with the lactose in the proportions given above. The blend is filled into hard gelatin capsules or cartridges or in specifically constructed double foil blister packs to be administered by an inhaler such as a Rotahaler, Diskhaler, or Diskus inhaler (each of these being a trademark of Glaxo Group Limited).

C Formulations for Nasal Administration

TABLE 3

| | |
|---|---|
| Cilomilast | 150 mg |
| Tiotropium bromide | 100 μg |
| Phenylethyl alcohol | 0.25 mL |
| Microcrystalline cellulose | 1.5 mg |
| and carboxymethylcellulose sodium (Avicel RC591) | |
| Benzalkonium chloride | 0.02 mg |
| Hydrochloric acid | to pH 5.5 |
| Purified water | to 100 mL. |

In a 100 μl metered volume dispensed by a Valois VP7 pre-compression pump, approximately 15 mcg of cilomilast and 10 mcg of tiopropium will be delivered.

D. Oral Tablet

Table 5 sets out a tablet formulation which can be used to administer a combination of PDE4 inhibitor and an anticholinergic agent.

TABLE 5

| Composition | Unit Formula |
|---|---|
| Cilomilast | 15.0 mg |
| Tiotropium | 36 μg |
| Lactose, Monohydrate | 99.64 mg |
| Microcrystalline Cellulose | 70.0 mg |
| Sodium Starch Glycolate | 10.0 mg |
| Magnesium Stearate | 2.0 mg |
| Total weight | 200.0 mg |

Tablet preparation is by conventional means using standard dry-powder mixing and a compression tableting tool.

What is claimed is:

1. A method of prophylaxis of, treating, or reducing the exacerbations associated with, a pulmonary disease by administering to a patient in need thereof an effective amount of a PDE 4 inhibitor and an anticholinergic agent either in a single combined form, separately, or separately and sequentially where the sequential administration is close in time, or remote in time.

2. A composition for the prophylaxis of, treating, or reducing the exacerbations associated with, a pulmonary disease comprising an effective amount of a PDE4 inhibitor, an effective amount of an anticholinergic agent, and a pharmaceutically acceptable excipient.

3. A method for preparing a composition which is effective for the prophylaxis of, treating, or reducing the exacerbations associated with, a pulmonary disease which method comprises mixing an effective amount of a PDE4 inhibitor and an anticholinergic agent with a pharmaceutically acceptable excipient.

4. The use of a composition comprising an effective amount of a PDE4 inhibitor, an effective amount of an anticholinergic agent and a pharmaceutically acceptable excipient in the manufacture of a medicament for the prophylaxis of, treating, or reducing the exacerbations associated with, a pulmonary disease.

5. An invention according to any one of the foregoing claims 1-4 in which the PDE4 inhibitor is cilomilast.

6. An invention according to any one of the foregoing claims 1-5 in which the anticholinergic agent is tiotropium or a salt thereof.

7. An invention according to any one of the foregoing claims 1-6 in which the PDE4 inhibitor is cilomilast and the anticholinergic agent is tiotropium or a salt thereof.

8. An invention according to any one of the foregoing claims 1-7 wherein the pulmonary disease is chronic obstructive pulmonary disease.

9. An invention according to any one of foregoing claims 1-7 wherein the pulmonary disease is asthma.
